(19) United States
(12) Patent Application Publication
(10) Pub. No.: US 2020/0030559 A1
(43) Pub. Date: Jan. 30, 2020

(54) EXPANDABLE ENDOTRACHEAL DEVICE

(71) Applicant: Aninimed, LLC, Chicago, IL (US)

(72) Inventors: Yuval AVNIEL, Missoula, MT (US); Kai MATTHES, Kula, HI (US)

(21) Appl. No.: 16/522,592

(22) Filed: Jul. 25, 2019

Related U.S. Application Data
(60) Provisional application No. 62/703,511, filed on Jul. 26, 2018.
(60) Provisional application No. 62/787,527, filed on Jan. 2, 2019.

(51) Int. Cl. A61M 16/04

(52) U.S. Cl. CPC A61M 16/0434; A61M 16/0463; A61M 16/0402

(57) ABSTRACT

An expandable endotracheal device includes: an elongated body configured to change from smaller-size state to a larger-size state; where in the smaller-size state, a perimeter, and thus a cross-sectional area, of the elongated body is restricted to facilitate insertion of the elongated body within a passageway of an in-use endotracheal tube; and where the elongated body is configured to: have at least a portion of the perimeter of the elongated body seal to a trachea of a subject; or provide an inner passageway extending through the elongated body along a length of the elongated body and with a boundary defining the inner passageway being configured to slidably receive a ventilation mechanism; or a combination thereof.

### CROSS-REFERENCE TO RELATED APPLICATIONS

This application claims the benefit of U.S. Provisional Application No. 62/703,511, filed Jul. 26, 2018, entitled “EXPANDABLE ENDOTRACHEAL TUBE,” and claims the benefit of U.S. Provisional Application No. 62/787,527, filed Jan. 2, 2019, entitled “AN EXPANDABLE CONDUIT FOR PRIMARY ENDOTRACHEAL INTUBATION OR ENDOTRACHEAL TUBE EXCHANGE,” with the entire contents of both of these applications being hereby incorporated herein by reference.

### BACKGROUND

While critically ill patients experience a life-threatening illness, they are also at risk from secondary conditions such as nosocomial infection. Pneumonia is the second most common nosocomial infection in critically ill patients, affecting approximately 27% of all critically ill patients. Eighty six percent (86%) of nosocomial pneumonias are associated with intubated patients, i.e., those that require mechanical ventilation with a breathing tube also referred to as endotracheal tube (ETT). Pneumonia that results due to intubation is commonly termed ventilator-associated pneumonia (VAP). In the United States alone, more than 300,000 cases of VAP occur per year, which is an incidence rate of between 5 to 10 cases per 1,000 hospital admissions. The mortality rate attributable to VAP has been reported to range up to 50% of those infected. Beyond mortality, the economics of VAP include increased intensive care unit (ICU) lengths of stays, which on average range from 4 to 13 days. Further the incremental costs associated with VAP have been estimated at between $5,000 and $20,000 per diagnosis.

For those patients requiring controlled ventilation, an endotracheal tube (ETT) is typically employed for insertion in a patient through the mouth for the purpose of ventilating the lungs. Insertion of an ETT may be through the mouth, the nasal passage, or direct access to the trachea via tracheotomy. The tube passes through the normally restricted glottis or passageway between the vocal cords and may terminate adjacent or near adjacent the bifurcation of the trachea into the right and left mainstem bronchus. Using an ETT that seals well to the trachea helps ensure oxygen and air is being delivered to the patient's lungs and helps prevent loss of oxygen/air from the ETT and delivery of oxygen/air to locations other than the lungs of the patient.

To accommodate patients having differently sized tracheas, a variety of endotracheal tubes of different diameters may be available to permit selection of the proper size tube for the patient. ETT may differ in size in length and/or diameter. ETTs with small cross-sectional diameters can result in insufficient or turbulent airflow delivery to the patient. Conversely, an ETT with too large a tube diameter may be difficult to pass the ETT through the vocal cords into the trachea, often resulting in trauma and/or complications for the patient, and may increase the time it takes to intubate the patient. Further, an ETT that is too large may also cause pressure on the trachea, which can result in tissue necrosis, inflammation, scarring and later on tracheal stenosis.

Patients requiring prolonged mechanical ventilation often require replacement of an ETT. The replacement of an ETT, referred to as re-intubation, may be performed by withdrawing the tube from the trachea and inserting a new tube into the trachea. This procedure is complicated, time consuming and incurs a high degree of risk to the patient. Further, re-intubation often requires a skilled hand to position a new ETT in the trachea properly. Due to the previous presence of the ETT in the airway for a short or longer duration of time, the tissue of the larynx, pharynx, and trachea often swells due to irritation and pressure on the tissue. This swelling creates a narrowing of the upper airway and makes the visualization of the vocal cords to re-insert the endotracheal tube more challenging than the initial intubation. Hence, there is often a reluctance for ICU physicians to extubate patients for the purpose of exchanging the ETT, And it is often very difficult to re-intubate the patient with a new ETT. This reluctance often results patients remaining intubated with the same ETT longer than anticipated, desired or wanted, which then increases the risk of VAP.

### SUMMARY

An example of an expandable endotracheal device includes: an elongated body configured to change from smaller-size state to a larger-size state; where in the smaller-size state, a perimeter, and thus a cross-sectional area, of the elongated body is restricted to facilitate insertion of the elongated body within a passageway of an in-use endotracheal tube; and where the elongated body is configured to: have at least a portion of the perimeter of the elongated body seal to a trachea of a subject; or provide an inner passageway extending through the elongated body along a length of the elongated body and with a boundary defining the inner passageway being configured to slidably receive a ventilation mechanism; or a combination thereof.

Implementations of such a device may include one or more of the following features. The elongated body provides, in the larger-size state, the inner passageway sufficiently sized for conveying fluid to lungs of the subject. The elongated body has a cross-sectional area of less than 60 mm² in the smaller-size state. The elongated body includes: (1) an inflatable bladder, (2) a stent, (3) a coil, (4) a shape-memory alloy, (5) a light-activated shape-changing material, (6) a mechanical expander, (7) an expandable material, or (8) a compression sleeve, or a combination of any of items (1)-(8). The elongated body is configured to provide different outward pressures at different positions along the length of the elongated body and/or to have different outer perimeter sizes at different positions along the length of the elongated body. The at least a portion of the perimeter of the elongated body is configured to adapt to a shape of a wall of the trachea of the subject. A distal end portion of the elongated body is curved along a length of the distal end portion. The elongated body includes a stent. The device includes a detachable ventilator connector detachably connected to a proximal end of the elongated body and configured to be connected to a ventilation system. The elongated body is configured to provide the inner passageway extending through the elongated body along the length of the elongated body and with the boundary defining the inner passageway being configured to slidably receive a ventilation mechanism, and wherein the elongated body comprises a plurality of rods extending lengthwise along the elongated body. The elongated body comprises a plurality of flexible membranes each connected to a pair of the rods along lengths of the rods in the pair of rods.

Another example of an expandable endotracheal device includes: means for conveying gas from a ventilation system to a lung of a subject; and means for altering at least one of a size or a shape of at least a portion of an outer perimeter of the means for conveying gas from a smaller-size state, in which the outer perimeter is sized and shaped to slide within a passageway of an in-use endotracheal tube, to a larger-size state in which at least a portion of the outer perimeter will seal a trachea of a subject.

Implementations of such a device may include one or more of the following features. The means for conveying gas are for providing an inner cavity through the means for conveying and sufficiently sized for conveying ventilation gas to lungs of the subject. The means for altering cause the means for conveying gas to have a cross-sectional area of less than 60 mm² in the smaller-size state. The means for altering include: (1) an inflatable bladder, (2) a stent, (3) a coil, (4) a shape-memory alloy, (5) a light-activated shape-changing material, (6) a mechanical expander, (7) an expandable material, or (8) a compression sleeve, or a combination of any of items (1)-(8). The means for altering are for providing different outward pressures at different positions along a length of the means for conveying and/or for providing different outer perimeter sizes at different positions along the length of the means for conveying. The means for altering are for adapting the at least a portion of the outer perimeter to a shape of a tracheal wall of the subject. A distal end portion of the means for conveying is curved along a length of the distal end portion. The means for altering include a stent. The means for conveying comprise a tube and a connector detachably connected to the tube and configured to be connected to the ventilation system. The means for conveying gas include a plurality of rods extending along a length of the means for conveying gas and a plurality of flexible membranes each attached to a pair of the plurality of rods. The means for altering at least one of the size or the shape of at least the portion of the outer perimeter of the means for conveying gas are configured to alter at least one of the size or the shape of at least the portion of the outer perimeter of the means for conveying gas in response to receipt of a member in a cavity defined by the means for conveying gas.

### BRIEF DESCRIPTION OF THE DRAWINGS

FIG. 1 is a perspective view of an example of an endotracheal tube.

FIGS. 2A-2C are perspective views of an example of an expandable endotracheal tube, that includes a compression sleeve, in a smaller-size state and two larger-size states, respectively.

FIGS. 3A-3B are perspective views of another example of an expandable endotracheal tube, that includes an expandable bladder, in a smaller-size state and a larger-size state, respectively.

FIGS. 3C-3D are cross-sectional views of the expandable endotracheal tube shown in FIGS. 3A-3B in the smaller-size state and the larger-size state, respectively.

FIGS. 4A-4B are perspective views of another example of an expandable endotracheal tube, that includes hinged panels, in a smaller-size state and a larger-size state, respectively.

FIGS. 4C-4D are cross-sectional views of a hinge of the expandable endotracheal tube shown in FIGS. 4A-4B in the smaller-size state and the larger-size state, respectively.

FIGS. 5A-5B are perspective views of another example of an expandable endotracheal tube, that includes an expandable coil, in a smaller-size state and a larger-size state, respectively.

FIGS. 6A-6B are perspective views of another example of an expandable endotracheal tube, that includes an expandable stent, in a smaller-size state and a larger-size state, respectively.

FIGS. 6C-6D are cross-sectional views of the expandable endotracheal tube shown in FIGS. 6A-6B in the smaller-size state and the larger-size state, respectively.

FIGS. 7A-7B are perspective views of another example of an expandable endotracheal tube, that includes pivotally connected tubular sections, in a smaller-size state and a larger-size state, respectively.

FIGS. 7C-7D are cross-sectional views of the expandable endotracheal tube shown in FIGS. 7A-7B in the smaller-size state and the larger-size state, respectively.

FIG. 7E is a close-up of a locking mechanism shown in FIG. 7D.

FIG. 8A is a side view of an expandable conduit in a compressed, small-size state.

FIG. 8B is a side view of the expandable conduit being in an intermediate-size state, expanded relative to the small-size state.

FIG. 8C is a side view of the expandable conduit being fully expanded, in large-size state, with a standard ETT positioned in a lumen of the expanded conduit.

FIG. 9A is a cross-sectional view of the expandable conduit in the compressed, small-size state.

FIG. 9B is a cross-sectional view of the expandable conduit in the intermediate-size state.

FIG. 9C is a cross-sectional view of the expandable conduit being fully expanded, in the large-size state, with a standard ETT positioned in the lumen of the expanded conduit.

FIG. 10A is a perspective view of the expandable conduit in the compressed, small-size state, positioned through a vocal cord of a patient with a tip in a tracheal lumen of the patient.

FIG. 10B is a perspective view of the expandable conduit being fully expanded, in the large-size state, positioned through the vocal cord with the tip in the tracheal lumen.

FIG. 10C is a perspective view of the expandable conduit being fully expanded, in the large-size state, with a standard ETT positioned in the lumen of the expanded conduit and advanced through the vocal cords into the tracheal lumen.

### DETAILED DESCRIPTION

Techniques are discussed herein for providing endotracheal devices with adjustable cross-sectional areas. For example, techniques are discussed for providing an ETT with an adjustable cross-sectional area over at least a portion or across the entire length of the ETT. An expandable endotracheal tube (EETT) has at least a portion thereof that is capable of expanding from a smaller cross-sectional area (e.g., corresponding to a smaller cross-sectional diameter) to a larger cross-sectional area (e.g., corresponding to a larger cross-sectional diameter). Also, techniques are discussed for providing intubation conduits with adjustable cross-sectional areas. An expandable endotracheal conduit (EEC) may be capable of expanding from a smaller cross-sectional area (e.g., corresponding to a smaller cross-sectional diameter) to a larger cross-sectional area (e.g., corresponding to a larger cross-sectional diameter). The conduit may be able to slide within an endotracheal tube (e.g., with the conduit having the smaller cross-sectional area) and may be able to slidably receive an endotracheal tube (e.g., with the conduit having the larger cross-sectional area).

Items and/or techniques described herein may provide one or more of the following capabilities, as well as other capabilities not mentioned. Insertion of endotracheal devices, such as endotracheal conduits and/or endotracheal tubes (e.g., fixed-cross-sectional area ETTs and/or expandable ETTs), may be facilitated. An expandable endotracheal device (EED), such as an EETT or an EEC, can be more easily inserted into a trachea than a non-expandable endotracheal device (such as a non-expandable ETT or a non-expandable endotracheal conduit), e.g., due to an initial smaller cross-sectional-area while being capable of being expanded to offer benefits of a larger endotracheal device. An EETT can replace an ETT presently used to ventilate a patient without ever losing control over the patient's trachea. An EETT can be used exchange a previously placed ETT while maintaining control of the airway of the patient. This method of exchanging the ETT may be associated with fewer risks commonly observed with prior re-intubation techniques, e.g., that require extubation, which means removal of the ETT and then re-intubation, which means insertion of a new ETT. Endotracheal tube exchange with from a regular ETT to an EETT can be performed while retaining the ETT within a patient's trachea during insertion of the EETT. An EETT may provide an improved seal between the ETT and a patient's trachea compared to prior ETTs, e.g., by increasing contact area between the patient's trachea and the ETT. Different pressures may be applied to a patient's trachea along a length of an ETT, e.g., with relatively lower pressures put on those parts of the trachea prone to damage due to pressure. Frequency of occurrence of VAP and other prolonged intubation associated issues may be reduced due to an improved seal between the upper airway, the oropharynx and the lower airways, the trachea. This helps prevent saliva from entering the lower airways. EETTs may be used for patient intubation without imparting excessive pressure or trauma, e.g., to a patient's vocal cords. EETTs may provide safer and/or less discomforting patient intubation than standard ETTs, and/or less tissue irritation during intubation than standard ETTs, e.g., due to smaller cross-sectional areas enabling easier passage within the patient (e.g., by the patient's vocal cords). An EETT may provide dynamic sealing between the EETT and a patient's trachea, and provide an interior lumen facilitating effective flow of respiratory gases through the EETT. An expandable conduit can be used to introduce an ETT into the trachea while passing the level of the vocal cords with little or no difficulty. For endotracheal tube exchange, an endotracheal conduit may be inserted into a previously-placed ETT. An endotracheal conduit may be used for the exchange of a previously-placed ETT while maintaining control of the airway of the patient. This method of exchanging the ETT may be associated with fewer risks commonly observed with prior re-intubation techniques. Endotracheal tube exchange with an endotracheal conduit may be performed while retaining the ETT within a patient's trachea during insertion of the new ETT. An ETT or EIC may be exchanged by persons other than doctors (e.g., anesthesiologists), nurses, or other highly skilled practitioners by using a technique of endotracheal tube exchange rather than re-intubation. The latter would involve direct or video laryngoscopy techniques through a skilled provider (i.e. anesthesiologists). Other capabilities may be provided and not every implementation according to the disclosure must provide any, let alone all, of the capabilities discussed

Provided herein are various configurations of expandable endotracheal devices (EEDs) and methods of making EEDs, including various configurations of expandable endotracheal tubes (EETTs) and expandable endotracheal intubation conduits (EICs) and methods of making and using EETTs and EICs. For example, a method for intubating a patient may include inserting an EETT into the patient's trachea and expanding a portion of the EETT effectively sealing the trachea such that effective intubation as is known in the art can ensue. Expanding the EETT may essentially conform at least a portion of the EETT to tracheal walls to seal the trachea. The EETT may adapt to the contours of the tracheal walls and/or the EETT may cause the tracheal walls to conform to the shape of the EETT such that the tracheal walls and an exterior of the EETT are in contact to facilitate positive pressure ventilation of the patient with an adequate seal. The EETT may apply moderate pressure to the patient's trachea to help seal the trachea, too much pressure can impede the perfusion of tissue. As another example, a method for intubating a patient may include inserting an expandable conduit into the patient's trachea (either directly or within an EET already disposed in the patient's trachea) and expanding the conduit to facilitate the introduction of an ETT (e.g., a new ETT) into the trachea. The term “patient” is used herein, but this does not imply that the discussion is limited to humans. The discussion herein may be applicable to use with a variety of subjects including humans, non-human animals, etc., and thus a “patient” could be any of such subjects.

EETTs as discussed herein may be anatomically shaped to fit within and seal a patient's trachea. EETTs may have exterior shapes similar to shapes of tracheal walls of patients.

EETTs as discussed herein may be configured not to impart undue pressure to zones within the human body that are not amenable to excess pressures. For example, EETTs discussed herein may be configured not to impart excessive pressure to vocal cords or excessive pressure on the mucosa of the trachea which might impede perfusion of the tissue.

An example EETT may include: a distal end section; an intermediate section connected to the distal end section; and a proximal end section including a ventilator connector that can be detachably connected between the EETT and a ventilation system, e.g., to a ventilator circuit, directly to a ventilation machine, etc. The proximal section may be connected to the intermediate section and the intermediate section may be capable of changing a cross-sectional area at least from a smaller cross-sectional area (e.g., a circular cross-sectional area with an outer diameter of 8 mm or less, 7 mm or less, 6 mm or less, of 5 mm or less, of 4 mm or less, or of 3 mm, or of 2 mm or less, or of 1 mm or less, or other shape of cross-section with similar corresponding areas) to a larger cross-sectional area (e.g., a circular cross-sectional area with an outer diameter of 10 mm or more, of 11 mm or more, or of 12 mm or more (or other shapes of similar areas), up to the maximum allowable outer diameter that is limited by an inner diameter of the trachea). Upon expansion, an expandable section forms a seal between the EETT and a patient's trachea walls and a lumen is provided along an interior length of the EETT with the lumen being capable of facilitating the passage of air flow to ventilate the patient. The ventilator connector may have a cross-sectional area that is smaller than a cross-sectional area of a lumen of an expanded EETT or of a standard ETT, or may be detachable such that when unattached an existing ETT or EETT can slide over the remaining portion of the EETT (e.g., the cross-sectional area of the remaining portion of the EETT is smaller than an inner diameter of the existing ETT or EETT). A special ventilator connector may include features that adjust to the variable size of the EETT in a way that a larger expanded EETT still provides enough seal between the ventilator connector and the inner diameter of the EETT. The seal can be created with a cone shaped design that slides in deeper into the lumen of the EETT with a larger diameter of expansion. Additional seal may be accomplished with a ring shaped constrictor that approximates the wall of the EETT to the ventilator connector.

Implementations of such an EETT may include one or more of the following features. The distal end portion may be shaped and sized as in typical endotracheal tubes. Alternatively, the distal end section may be capable of expansion and may be configured similarly to the intermediate section.

Implementations of such an EETT may include one or more of the following features. The intermediate section may include an inflatable bladder for changing the cross-sectional area. The distal end section may also include an inflatable bladder (or part of the inflatable bladder of the intermediate section) that is capable of changing a cross-sectional area of the distal end section from a smaller area to a larger area. The inflatable bladder may be connected to a guide that may be used to help position the EETT at an appropriate position within a patient's trachea. The bladder itself may have sufficient structural rigidity, and appropriate shape and length to act as a guide for positioning the EETT properly in a patient's trachea. The inflatable bladder may be capable of expanding such that the bladder abuts the tracheal walls, effectively sealing against the tracheal walls and inhibiting leak of air or gases between the tracheal walls and the outside of the bladder. The bladder may have an orifice extending the length of the bladder such that the bladder, when expanded, is a tube such that ventilated air or gases can flow through the bladder. The bladder may be anatomically shaped (e.g., shaped similarly to tracheal walls) to fit within and seal a patient's trachea. The bladder may expand to different cross-sectional areas and/or may apply different pressures to specific parts of a patient's trachea. The bladder may have a valve configured to retain inflation gas within the bladder. For example, the valve may be a one-way valve, a two-way valve, a valve that can be opened and/or closed, a valve that is always open, etc.

Also or alternatively, implementations of an EETT may include one or more of the following features. The intermediate section may include a stent-like architecture for expanding the cross-sectional area. The distal end section may include a stent-like architecture for changing a cross-sectional area of the distal end section. The distal end section and the intermediate end section may be formed from the same stent like architecture. The stent-like architecture may provide an orifice or lumen along a length of the stent-like architecture such that, upon expansion, the architecture is tubular shaped such that ventilated air or gases can flow within the orifice or lumen. The proximal end section, the intermediate section, and the distal end section may include a stent-like architecture.

Also or alternatively, implementations of an EETT may include one or more of the following features. The intermediate section may include a locking mechanism capable of locking the expandable sections such that after expansion, the intermediate section is inhibited from reducing the cross-sectional area.

Also or alternatively, implementations of an EETT may include one or more of the following features. The intermediate section may be configured to change the cross-sectional area by removal of a compression system that limits expansion of the intermediate section. The distal end section may also be configured to change a cross-sectional area of the distal section by removal of a compression system that limits expansion of the distal end section.

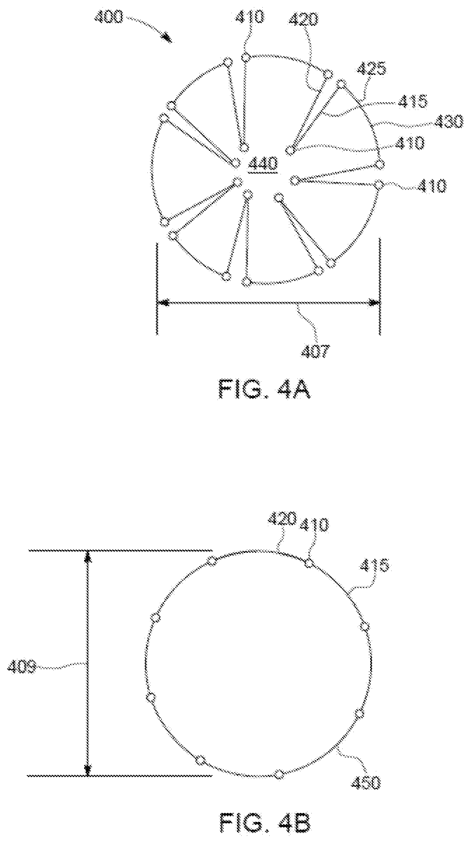

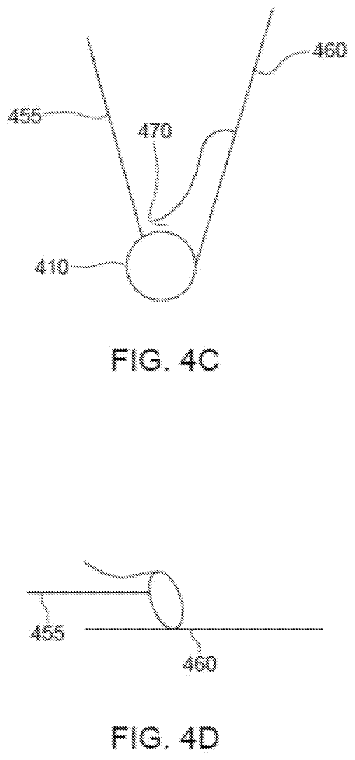

Also or alternatively, implementations of an EETT may include one or more of the following features. The intermediate section may include hinged panels for changing the cross-sectional area. For example, the hinged panels, in the smaller cross-sectional area state, alternate between one panel forming part of small outer perimeter and two panels extending from the outer perimeter inside of the outer perimeter (see FIG. 4A). Further in this example, the hinged panels, in the larger cross-sectional area state, each constitute part of a large outer perimeter (see FIG. 4B) and form a tube with an interior lumen along the length of the EETT that enables intubation. The distal end section may also include hinged panels capable of changing a cross-sectional area of the distal end section in a manner similar to the hinged panels of the intermediate section.

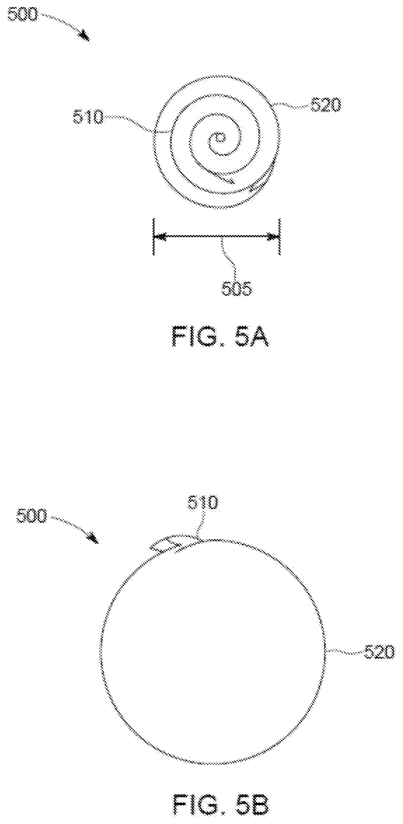

Also or alternatively, implementations of an EETT may include one or more of the following features. The intermediate section may include nested spirals (see FIG. 5A for changing the cross-sectional area. The nested spirals may be rotated to expand the cross-sectional area of the EETT and to provide a lumen along the length of the EETT that enables ventilation. The distal end section may also include nested spirals capable of changing a cross-sectional area of the distal end section in a manner similar to the nested spirals of the intermediate section. An inner-most spiral may applies an outward pressure to the other spiral(s). A mechanism may be included that is configured to lock spirals (e.g., the inner-most spiral and the outer-most spiral) to each other to inhibit reduction their cross-sectional areas once expanded.

Also or alternatively, implementations of an EETT may include one or more of the following features. One or more sections of an ETT may assume a relatively small cross-sectional area for insertion into a patient's trachea and/or an existing endotracheal tube and be expanded to seal an interface between the EETT and the trachea sufficiently to facilitate ventilation of the lungs.

Also or alternatively, implementations of an EETT may include one or more of the following features. The intermediate section may be configured to expand from a small cross-sectional area to a larger cross-sectional area at the discretion of a user. The distal end section may be used to facilitate insertion of the EETT and/or positioning of the EETT and the distal end may be connected to the proximal end section of the EETT by a material of sufficient rigidity that the distal end can be passed through a patient's trachea to an appropriate position in the (center of) the trachea with sufficient distance from the vocal cords and distance from the carina (i.e., the bifurcation of the trachea). Many materials offer sufficient rigidity and softer materials can be formed such that they offer sufficient rigidity. Examples of materials that may be used include, but are not limited to, polyvinyl chloride, silicone, rubber, or polyurethane. This list of suitable materials provided is not an exhaustive list and other medical-grade, non-allergenic materials may be used, whether presently existing or developed in the future. The rigidity of the positioning mechanism/material (of the distal end section) can be greater than the rigidity of the remainder of the EETT. Further, different parts of the EETT can have different rigidities. The EETT may be configured to inhibit, or even prevent, kinking, e.g., by comprising a sufficiently rigid material and/or by providing a sufficient radial force.

Also or alternatively, implementations of an EETT may include one or more of the following features. The distal end section may not be more rigid than other sections of the EETT. The distal end section may be attached to the intermediate section by any suitable means, such as by an adhesive, heat sealing, solvent bonding, RF (radio frequency) sealing, or ultrasonic welding, etc. The distal end section and the other sections of the EETT may be formed as a single integral system out of one or more materials. The EETT may be made in a variety of ways, e.g., by being blow-molded, three-dimensionally printed, sprayed, extruded, or assembled by means that enable use of disparate materials. For example, with blow molding, the intermediate section may have relatively thinner walls than the distal end section and/or the proximal end section. The distal end section may be cylindrical and have outer diameter of the distal end section, when expanded, may be between 1 mm and 14 mm or greater. A range of sizes of the distal end section may facilitate effective intubation for patients with a variety of different trachea sizes, e.g., for pediatric applications or for adult applications.

Also or alternatively, implementations of an EETT may include one or more of the following features. The distal end section may be any suitable length from the proximal end section. For example, the distal end section may be 6-30 centimeters or greater from the proximal end section, although other separation distances may be used. For instance, veterinary endotracheal tubes may be meters in length and function as described herein, or substantially smaller for small animal research using e.g. rodents. The distal end section may include an opening in a sidewall of the distal end section near a distal end of an inner lumen provided by the distal end section. This opening is commonly called a “Murphy eye” and may serve as alternative path in the event that the distal opening becomes blocked. The distal end section may be beveled and/or rounded to allow for smoother insertion through the larynx and trachea. The distal end section and/or apex of the distal end section may be made of a pliable material to lessen the likelihood of trauma produced by insertion of the distal end section through the trachea.

Also or alternatively, implementations of an EETT may include one or more of the following features. The proximal end section may be formed from conventional plastics or polymers, including medical-grade materials such as polyvinyl chloride. The proximal end section may be attached to the intermediate section by any suitable means, such as by adhesives, heat sealing, etc. The proximal end section and the intermediate section may be integral with one or more other portions of the EETT. Two or more sections of the EETT may be formed in combination.

Also or alternatively, implementations of an EETT may include one or more of the following features. The proximal end section may include a mechanism (a coupling device or attachment means) capable of being attached to and detached from another device or system such as a ventilator or other medical device. For example, the proximal end section may include a quick-disconnect coupling, a standard 15 mm outer diameter coupling, or a ventilation coupler. The proximal end section may have an outer diameter that is between approximately 2 mm and 12 mm. The size of the outer diameter may depend on a size of the patient, e.g., whether the patient is a pediatric patient or an adult patient. The proximal end section may, however, be larger than 12 mm, e.g., for veterinary uses. The proximal end section maybe any suitable length such as between about 0.1 cm and about 50 cm for human intubation, or longer, as appropriate, for veterinary applications. Still other lengths may be used, including expandable sections of meters in length or overall lengths of meters.

Also or alternatively, implementations of an EETT may include one or more of the following features. The proximal end section may be manipulated by medical staff during tube insertion and connection. At least a portion of an outer perimeter may be configured for connection to ventilator tubing. For example, an outer diameter of the proximal end section may be about 10-20 mm or greater. Such a diameter may allow direct connection of the proximal end section to ventilator tubing or to one or more connection pieces, providing multiple connection options.

Also or alternatively, implementations of an EETT may include one or more of the following features. An EETT may provide any suitable number of lumens that may be appropriately sized and shaped for intubation, suction, or other conveyance of a fluid (e.g., a gas or a liquid), e.g., for medical procedures.

Also or alternatively, implementations of an EETT may include one or more of the following features. One or more of the intermediate section, the proximal end section, or the distal end section is configured to have an expandable cross-sectional area (e.g., corresponding to an expandable cross-sectional diameter). A size of the expandable section(s) may be reduced to fit inside of (e.g., slide inside of) an ETT or EETT. For example, the outer diameter(s) of the expandable section(s) may be smaller than an inner diameter of an ETT or EETT, with the inner diameter measured from interior wall to interior wall of the ETT or EETT perpendicular to the walls and perpendicular to a length of the ETT or EETT into which the expandable section(s) is(are) inserted and with the outer diameter measured from exterior wall to exterior wall perpendicular to the respective walls and/or perpendicular to a length of the of the respective expandable section. A maximum expandable cross-sectional area (e.g., corresponding to a maximum outer diameter) may be larger than a tracheal diameter before insertion of the EETT. While inserted into a patient, the outer perimeter of the expandable portion may generally conform to the trachea walls. The EETT may be configured to apply less pressure to the trachea, vocal cords, or other feature than will cause damage. The expandable section(s) may be configured such that the external pressure from the expandable section(s) applied to the trachea will not exceed the perfusion pressure of mucosal tissue, thereby avoiding decreased perfusion of the tissue which could result in necrosis and later on scarring or stenosis. For example, the expandable section(s) may be configured to apply less than 60 mm Hg of pressure, less than 50 mm Hg of pressure, less than 40 mm Hg of pressure, less than 30 mm Hg of pressure, or less than 20 mm Hg of pressure, or less than 10 mm Hg of pressure. An EETT may be configured to be narrower, having a smaller cross-sectional area (e.g., smaller diameter) in a region of a patient's vocal cords than other regions thus limiting trauma or pressure on the vocal cords which can result in voice problems, recurrent laryngeal nerve damage, development of nodules on the vocal cords, and damage to the vocal cord cartilage.

An example method for sealing a patient's trachea includes: inserting an EETT into the patient's trachea and/or into an existing ETT in the patient's trachea and/or into an existing EETT in the patient's trachea; removing the existing ETT or existing EETT if present; and expanding the EETT such that the EETT forms a seal with tracheal walls of the patient's trachea. The EETT may substantially conform to the tracheal walls and/or other anatomical structure such that the EETT applies different pressures to different portions of the patient, e.g., less pressure to anatomical features that are more easily damaged due to pressure.

Also provided herein are various configurations of expandable intubation conduits and methods of making and using expandable intubation conduits for primary endotracheal intubation and endotracheal tube exchange. For example, a method for intubating a patient may include inserting an expandable conduit into the patient's trachea, expanding the conduit to facilitate the introduction of the ETT into the trachea, inserting the ETT into the EIC and thus into the trachea, and removing the EIC. The term “patient” is used herein, but this does not imply that the discussion is limited to humans. The discussion herein may be applicable to use with a variety of subjects including humans, non-human animals, etc., and thus a “patient” could be any of such subjects.

An expandable intubation conduit (EIC) may include: a distal end section; an intermediate section connected to the distal end section; and a proximal end section including a ventilator connector that may, for example, be detachably connected between the EIC and a ventilation system, e.g., to a ventilator circuit, directly to a ventilation machine, etc. The proximal section may be connected to the intermediate section and the intermediate section may be capable of changing a cross-sectional area at least between a smaller cross-sectional area (e.g., a circular cross-sectional area with a diameter of 6 mm or less, of 5 mm or less, of 4 mm or less, or of 3 mm, or of 2 mm or less, or of 1 mm or less) and a larger cross-sectional area (e.g., a circular cross-sectional area with a diameter of 10 mm or more, of 11 mm or more, or of 12 mm or more, up to the maximum allowable diameter that is limited by the diameter of the trachea). Upon expansion, the EIC may form a seal between the EIC and a patient's trachea walls. A lumen may be provided along an interior length of the EIC with the lumen being capable of facilitating the passage of air to ventilate the patient. The ventilator connector may have a cross-sectional area that is smaller than a cross-sectional area of a lumen of an expandable conduit or of a standard ETT, and/or may be detachable from the ETT. The ventilator connector may include features that adjust to the variable size of the expandable conduit in such a way that for a larger expanded conduit, the connector still provides enough seal between the ventilator connector and the inner diameter of the expandable conduit to enable adequate patient ventilation through the conduit (e.g., without impermissibly high leakage). For example, a pressure (sometimes called an insperitory pressure) at least about 20 mm of mercury, e.g., 20-30 mm Hg or even 40 mm Hg or more for lungs impeding inflation (e.g., due to illness), may be used to ventilate a patient. The seal can be created with a cone-shaped member that may slide deeper than a ring-shaped connector into the lumen of the EIC with a larger diameter of expansion (i.e., the cone-shaped member, having many different diameters along a length of the cone-shaped member, may seal to a wider range of lumen cross-sectional areas than a ring-shaped member). Additional seal may be accomplished with a ring-shaped constrictor that approximates the wall of the EIC to the ventilator connector, e.g., a ring that slides over the connector.

Implementations of such an EIC may include one or more of the following features. The distal end portion may be shaped and sized as in typical endotracheal tubes, e.g., with a bevel and a Murphy eye (discussed below). Alternatively, the distal end section may be capable of expansion and may be configured similarly to the intermediate section.

Also or alternatively, implementations of such an EIC may include one or more of the following features. The intermediate section may include longitudinal rods and soft membranes between the rods. The rods may be flexible so that they can adjust to the anatomy of the patient while providing enough stiffness so that the ETT can slide inside the conduit without getting caught at the level of the vocal cords or other narrowing of the airway. Various quantities of stiffening rods may be used, e.g., between 1 and 20, or even more than 20. The membranes may be flexible and may be configured to expand with the conduit from a small diameter lumen to a larger diameter. The membranes may be made of silicone or latex or other flexible material.

The membranes may facilitate a change of the cross-sectional area. The distal end section may also include a flexible membrane (or part of the intermediate section) that is capable of changing a cross-sectional area of the distal end section between a smaller area to a larger area. The EIC may be connected to a guide that may be used to help position the EIC at an appropriate position within a patient's trachea. The EIC may have sufficient structural rigidity, and appropriate shape and length to act as a guide for positioning the EIC properly in a patient's trachea. The EIC may be capable of expanding such that the outer diameter abuts the tracheal walls of the patient, effectively sealing against the tracheal walls and inhibiting leakage of air or gases between the tracheal walls and the outside of the EIC (e.g., an expandable tube or expandable cuff).

The EIC may have an orifice extending the length of the EIC that, when expanded, is a tube such that ventilated air or gases can flow through the conduit. The EIC, with its flexible structure, may autonomously conform to fit within and seal a patient's trachea (e.g., become shaped similarly to tracheal walls). The EIC may expand to different cross-sectional areas (sizes and/or shapes) and/or may apply different pressures to specific parts of a patient's trachea. The EIC may have a valve configured to retain inflation gas within the conduit. For example, the valve may be a one-way valve, a two-way valve, a valve that can be opened and/or closed, a valve that is always open, etc.

Also or alternatively, implementations of an EIC may include one or more of the following features. One or more sections of an ETT may assume, or be made to have, a relatively small cross-sectional area for insertion into a patient's trachea and/or an existing endotracheal tube and may expand, or be made to expand, to seal an interface between the EIC and the trachea sufficiently to facilitate ventilation of the lungs.

Also or alternatively, implementations of an EIC may include one or more of the following features. The intermediate section may be configured to change between a small cross-sectional area and a larger cross-sectional area (e.g., change from the small cross-sectional area to the larger cross-sectional area and vice versa) at the discretion of a user. The distal end section may be used to facilitate insertion of the EIC and/or positioning of the EIC and the distal end may be connected to the proximal end section of the EIC by a material of sufficient rigidity that the distal end can be passed through a patient's trachea to an appropriate position in the (center of the) trachea with sufficient distance (e.g., about 5-10 mm) further into the trachea from the vocal cords and the carina (i.e., the bifurcation of the trachea) to avoid damage to the vocal cords or carina. The conduit may be made of a material with sufficient rigidity to avoid kinking or bending of the sleeve conduit. Examples of materials that may be used include, but are not limited to, polyvinyl chloride, silicone, rubber, or polyurethane. This list of suitable materials provided is not an exhaustive list and other medical-grade, non-allergenic materials may be used, whether presently existing or developed in the future. The rigidity of the positioning mechanism/material (of the distal end section) may be greater than the rigidity of the remainder of the EIC. Further, different parts of the EIC can have different rigidities. The EIC may be configured to inhibit, or even prevent, kinking, e.g., by comprising a sufficiently-rigid material and/or by providing a sufficient radial force.

Also or alternatively, implementations of an EIC may include one or more of the following features. The distal end section may be of a similar rigidity as other sections of the EIC. The distal end section may be attached to the intermediate section by suitable means, such as by an adhesive, heat sealing, solvent bonding, RF (radio frequency) sealing, or ultrasonic welding, etc. The distal end section and the other sections of the EIC may be formed as a single integral system out of one or more materials. The EIC may be made in a variety of ways, e.g., by being blow-molded, three-dimensionally printed, sprayed, extruded, or assembled by means that enable use of disparate materials. For example, with blow molding, the intermediate section may have relatively thinner walls than the distal end section and/or the proximal end section. The distal end section may be cylindrical and may have an outer diameter, when expanded, between 1 mm and 14 mm or greater. A range of sizes of the distal end section may facilitate effective intubation for patients with a variety of different trachea sizes, e.g., for pediatric applications or for adult applications.

Also or alternatively, implementations of an EIC may include one or more of the following features. The distal end section may be any suitable length from the proximal end section. For example, the distal end section may be between 6 and 30 centimeters from the proximal end section, although other separation distances may be used (e.g., further than 20 cm). For instance, veterinary endotracheal tubes may be meters in length and function as described herein, or substantially smaller for small animal research, e.g., using rodents. The distal end section may include an opening in a sidewall of the distal end section near a distal end of an inner lumen provided by the distal end section. This opening is commonly called a “Murphy eye” and may serve as an alternative path in the event that the distal opening becomes blocked. The distal end section may be beveled and/or rounded to allow for smoother insertion through the larynx and trachea. The distal end section and/or an apex of the distal end section may be made of a pliable material to lessen the likelihood of trauma produced by insertion of the distal end section through the trachea.

Also or alternatively, implementations of an EIC may include one or more of the following features. The proximal end section may be formed from conventional plastics or polymers, including medical-grade materials such as polyvinyl chloride. The proximal end section may be attached to the intermediate section by any suitable means, such as by adhesives, heat sealing, etc. The proximal end section and the intermediate section may be integral with one or more other portions of the EIC. Two or more sections of the EIC may be formed in combination.

Also or alternatively, implementations of an EIC may include one or more of the following features. The proximal end section may include a mechanism (e.g., a coupling device or attachment means) capable of being attached to and detached from another device or system such as a ventilator or other medical device. For example, the proximal end section may include a quick-disconnect coupling, a standard 15 mm outer diameter coupling, or a ventilation coupler. The proximal end section may have an outer diameter that is between approximately 2 mm (e.g., 2 mm+/−10%) and 12 mm (e.g., 12 mm+/−10%). The size of the outer diameter of the EIC selected for use may depend on a size of the patient, e.g., whether the patient is a pediatric patient or an adult patient. The proximal end section may, however, be larger than 12 mm, e.g., for veterinary uses. The proximal end section may be any suitable length such as between about 0.1 cm (e.g., 0.1 cm+/−10%) and about 50 cm (e.g., 50 cm+/−10%) for human intubation, or longer, as appropriate, for veterinary applications. Still other lengths may be used, including expandable sections of meters in length or overall lengths of meters.

Also or alternatively, implementations of an EIC may include one or more of the following features. The proximal end section may be configured to mate with another device and/or to change (or be changed) in size, e.g., by medical staff during tube insertion and connection. At least a portion of an outer perimeter may be configured for connection to ventilator tubing. For example, an outer diameter of the proximal end section may be about 10-20 mm or greater. Such a diameter may allow direct connection of the proximal end section to ventilator tubing or to one or more connection pieces, providing multiple connection options.

Also or alternatively, implementations of an EIC may include one or more of the following features. An EIC may include a suitable number of lumens that are appropriately sized and shaped for intubation, suction, or other conveyance of a fluid (e.g., a gas or a liquid), e.g., for medical procedures.

Also or alternatively, implementations of an EIC may include one or more of the following features. One or more of the intermediate section, the proximal end section, or the distal end section is configured to have an expandable cross-sectional area (e.g., corresponding to an expandable cross-sectional diameter). The size(s) of the expandable section(s) may be set (e.g., reduced) to fit inside of (e.g., slide inside of) an ETT. For example, the outer diameter(s) of the expandable section(s) may be smaller than an inner diameter of an ETT or EIC, with the inner diameter measured from interior wall to interior wall of the ETT or EIC perpendicular to the walls and perpendicular to a length of the ETT or EIC into which the expandable section(s) is(are) inserted and with the outer diameter measured from exterior wall to exterior wall perpendicular to the respective walls and/or perpendicular to a length of the of the respective expandable section. A maximum expandable cross-sectional area (e.g., corresponding to a maximum outer diameter) may be larger than a tracheal diameter before insertion of the EIC. While inserted into a patient, the outer perimeter of the expandable portion may generally conform to the trachea walls. The EIC may be configured to apply less pressure to the trachea, vocal cords, or other feature than will cause damage. The expandable section(s) may be configured such that the external pressure from the expandable section(s) applied to the trachea will not exceed the perfusion pressure of mucosal tissue, thereby avoiding decreased perfusion of the tissue which could result in necrosis and later on scarring or stenosis. For example, the expandable section(s) may be configured to apply less than 60 mm Hg of pressure, less than 50 mm Hg of pressure, less than 40 mm Hg of pressure, less than 30 mm Hg of pressure, or less than 20 mm Hg of pressure, or less than 10 mm Hg of pressure. An EIC may be configured to be narrower, having a smaller cross-sectional area (e.g., smaller diameter) in a region of a patient's vocal cords than other regions thus limiting trauma or pressure on the vocal cords which can result in voice problems, recurrent laryngeal nerve damage, development of nodules on the vocal cords, and/or damage to the vocal cord cartilage.

An example method for sealing a patient's trachea includes: inserting an EIC into the patient's trachea and/or into an existing ETT in the patient's trachea and/or into an existing EIC in the patient's trachea; removing the existing ETT or existing EIC if present; and expanding the EIC such that the EIC forms a seal with tracheal walls of the patient's trachea. The EIC may substantially conform to the tracheal walls and/or other anatomical structure such that the EIC applies different pressures to different portions of the patient, e.g., less pressure to anatomical features that are more easily damaged due to pressure.

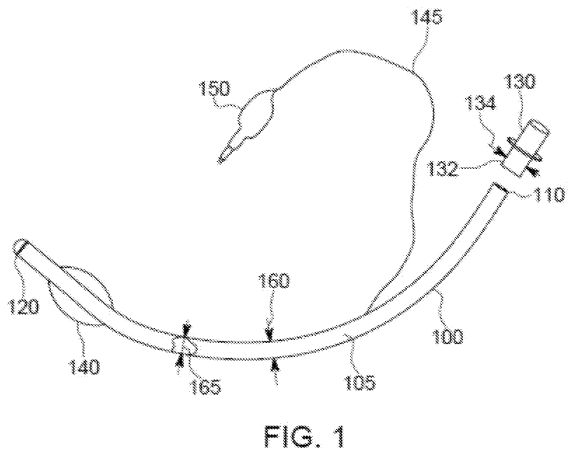

Referring now to FIG. 1, an example of an expandable endotracheal device, here an example of an endotracheal tube 100, includes a body 105 having a proximal end 110 and a distal end 120, a cuff 140, a pilot tube 145, and a pilot tube balloon 150. The proximal end 110 is configured (e.g., sized, shaped) to accommodate a ventilator connector 130 that is detachable from the proximal end 110. The ventilator connector 130 has a distal end 132 with an outer diameter 134 (and corresponding inner diameter) sized to receive the proximal end 110 of the body 105. The cuff 140, sometimes referred to as a balloon cuff, is commonly disposed near the distal end 120, e.g., about 1-10 mm from the distal end 120. The pilot tube 145 and the pilot tube balloon 150 are optional but included in the ETT 100 in this example. The pilot tube balloon 150 may be attached to the body 105 by a user. The ETT 100 is an EETT, and so is capable of having a cross-sectional area 160 expanded from a smaller cross-sectional area to a second larger cross-sectional area (e.g., corresponding to smaller and larger inner diameters 165), e.g., as described herein. The smaller cross-sectional area is of a size and shape such that the ETT 100 will fit within an ETT already located in the tracheal lumen and used to ventilate a subject while the larger cross-sectional area is of a size and shape, and the ETT 100 is configured, such that the ETT 100 can expand to provide a seal with tracheal walls and to provide a lumen amendable to intubation (or ventilation).

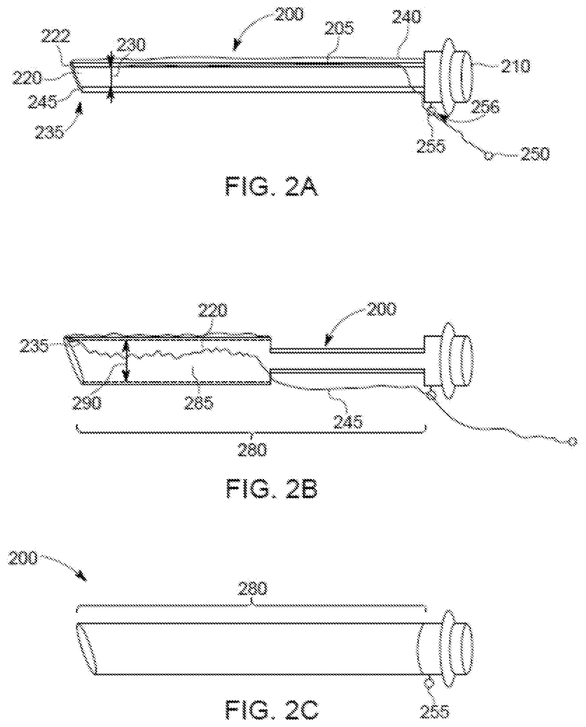

Referring to FIGS. 2A-2C, an example EETT 200 includes a ventilator connector 210, a compression sleeve 220, a release cord 245, and a guide ring 255. The EETT 200 is configured to change between a smaller-size state (e.g., an insertion state that may facilitate insertion) as shown in FIG. 2A and a larger-size state (e.g., an expanded state that may facilitate intubation) as shown in FIG. 2C. As shown in FIG. 2A, the compression sleeve 220 is configured to compress an expandable portion of the EETT 200 to a relatively smaller cross-sectional area, here corresponding to a relatively smaller diameter 230. Upon removal of the compression sleeve 220 (as shown in FIGS. 2B-2C), the expandable portion of the EETT 200 expands to a larger cross-sectional diameter. The compression sleeve 220, in this example, spans from a distal end 235 of the EETT 200 to a proximal end 240. The ventilator connector 210 is located at the proximal end 240 and may be detachable from the proximal end 240 or fixedly attached to the proximal end 240. The release cord 245 is detachably connected to the EETT 200 at the proximal end 240, i.e., at or near an intersection of the EETT 200 and the ventilator connector 210. The release cord 245 is configured to release the compression sleeve 220 from compressing the expandable portion of the EETT 200 to release the expandable portion, allowing the expandable portion to expand. Other forms of release mechanisms, i.e., other than a cord as shown, may be used.

The release cord 245 is disposed and configured to tear the compression sleeve 220 in order to release it from compressing the expandable portion of the EETT 200. The release cord 245 is attached to a body 205 of the EETT 200 near the proximal end 240 and extends from the proximal end 240 along a length of the EETT 200 between the body 205 and the compression sleeve 220, around a distal end 222 of the compression sleeve 220, and back to the proximal end 240. The release cord 245 extends through an eyelet 256 provided in a guide ring 255 sufficiently far such that an end 246 of the release cord 245 is accessible to a user (e.g., can be firmly grasped by a hand or a tool). The release cord 245 is releasably held in place such that the end 246 of the release cord 245 is stationary or nearly so. In this example, the end 246 includes a member 250 (e.g., a tag, knob, etc.) configured to facilitate grasping by the user (e.g., by the hand or the tool). The guide ring 255 helps define a travel path of the release cord 245 to help ensure that pulling of the release cord will tear the compression sleeve 220, although the guide ring 255 is optional and another mechanism may be used to guide the release cord 245 (or no guide provided for the release cord 245). The release cord 245 abuts an outside of the compression sleeve 220. Alternatively, the release cord 245 could be encased within an aperture of guide mechanism positioned against or within the compression sleeve 220 (which may be referred to as a retaining sleeve). The guide mechanism could comprise a single member or a combination of members (e.g., separate loops), and could extend the length of the compression sleeve 220. The use of the guide mechanism through which the release cord 245 travels may facilitate travel of the release cord 245 while inhibiting adverse interactions between the release cord 245 and the patient.

With particular reference to FIG. 2B, the EETT 200 is in a partially expanded state, with the release cord 245 having been pulled such that approximately half of the compression sleeve 220 has been torn (released) such that approximately half of an expandable portion 280 of the EETT 200 has been expanded due to the ripping of the compression sleeve 220. An expanded portion 285 of the EETT 200 has been expanded to a cross-sectional diameter 290 that is larger than the diameter 230 and is predefined by a configuration of the expandable portion user or until the expanded portion 285 is constrained (e.g., by tracheal walls) to an area that is smaller than a maximum area of the expanded portion 285. The expandable portion 280 is configured such that an amount of expansion and a pressure providable by the expandable portion 280 (or sub-portions thereof) are limited as desired, e.g., to within medically appropriate levels as discussed herein. Pulling the release cord 245 results in tearing the compression sleeve 220 thereby enabling the compressed EETT 200 to expand wherever the compression sleeve 220 has been torn. Tearing the compression sleeve 220 is actuated by pulling the release cord 245 through the eyelet 256 and thus pulling the release cord through the compression sleeve 220. In this example, the tearing begins at the distal end 222 of the compression sleeve 220 and progresses towards the proximal end 240 as the release cord 245 is pulled. In some embodiments, the compression sleeve 220 is perforated or otherwise weakened along a line of travel of the release cord 245 to facilitate separation of the retaining sleeve 220 upon actuation from the release cord 245. Such perforations may be positioned such that they coincide with the positioning of the release cord 245.

With particular reference to FIG. 2C, the EETT 200 is in a fully-expanded state, with the release cord 245 having been pulled such that all of the compression sleeve 220 has been torn (released, and possibly removed) such that all of the expandable portion 280 of the EETT 200 has been expanded due to the ripping of the compression sleeve 220. The expanded portion 285 of the EETT 200 has been expanded to the cross-sectional diameter 290. In the example shown, an outer perimeter of the expandable portion 280 is essentially the same as an outer perimeter of the ventilator connector 210, but other size/shape relationships may be used. For example, the outer perimeter of the expandable portion 280 may be larger than the outer perimeter of the ventilator connector 210. Also or alternatively, the compression sleeve 220 may be internal to the EETT 20.

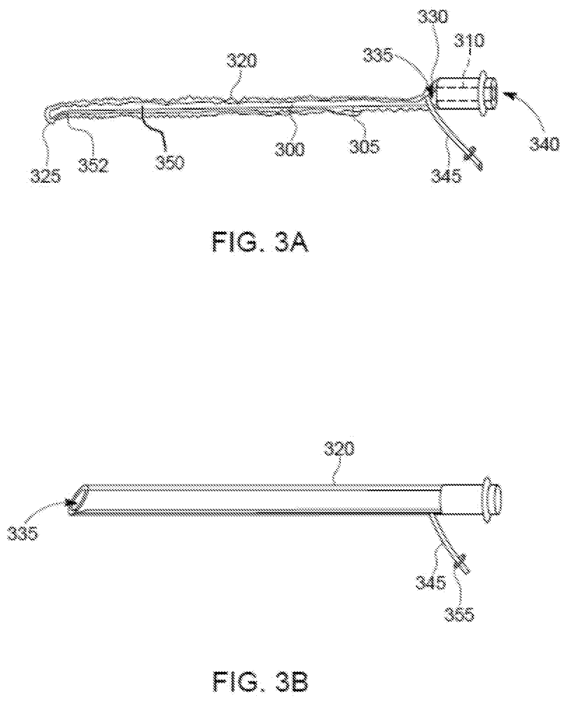

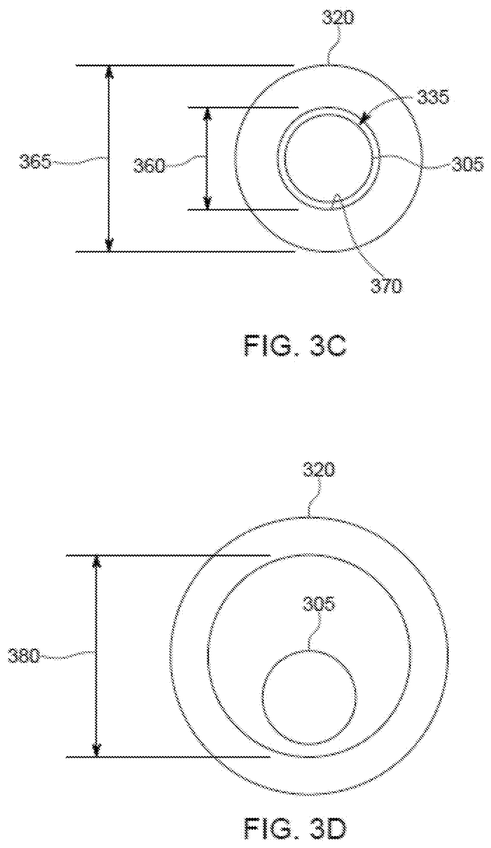

Referring to FIGS. 3A-3D, another example EETT 300 includes a body 305, a ventilator connector 310, and an expandable bladder 320. Here, the bladder 320 is a tubular bladder that is configured to be inflated. The ventilator connector 310 is connected to the body 305 and may be configured to be attached to and detached from the body 305, e.g., at the discretion of a user. The ventilator connector 310 may be, for example, a 22 mm outside diameter connector, and may be made by, for example, Tri-Amin of Dublin, Ohio, or Hamilton Medical of Bonaduz, Switzerland. Here, the body 305 is configured as a thin positioning bar extending from the ventilator connector 310 to a distal end 325 of the EETT 300. The EETT 300 may be airtight such that gas inside the EETT 300 is prevented from exiting the EETT 300 except at an end of the EETT 300.

The body 305 may be specifically configured (e.g., sized and/or shaped) for a subject into which the body 350 is to be inserted. The body 305, or the EETT 300 as a whole, may be configured to be gentle to a subject in which the EETT 300 is inserted. The body 305 may be configured for the anatomy of a subject, e.g., having a curved portion for insertion into a human trachea and/or for comfortable insertion and/or final positioning in the subject. For example, the body 305 may include a curve 352 at the distal end 325 that is configured to facilitate positioning the distal end 325 of the EETT 300 in a desired position in a patient. Also or alternatively, the body 305 may be curved between ends of the body 305, e.g., similar to a curve in a patient from mouth to trachea. Also or alternatively, the body 305 may be configured (e.g., composed of a material and/or shaped) for comfort under pressure, e.g., may compress in response to a subject swallowing while the body 305 is in the subject. Also or alternatively, the body 305 may be configured to apply different pressures at different points along a length of the body 305, e.g., due to different cross-sectional sizes along the length. The body 305 may be configured to apply the different pressures to different anatomical features (e.g., vocal cords vs. trachea wall) while applying less pressure to more sensitive areas.

The body 305 is connected to the bladder 320 at the distal end 325 of the EETT 300. The body 305 is disposed in an orifice 335 provided by the bladder 320 that extends the length of the bladder 320 from the distal end 325 of the EETT 300 to a proximal end 330 of the body 305. The bladder 320 connects to the ventilator connector 310 such that the orifice 335, at the proximal end 330, coincides with an orifice 340 provided by, and extending through, the ventilator connector 310. The bladder 320 includes an inlet 345 through which a fluid, e.g., a gas or other material, can be introduced to the bladder 320 to expand the bladder 320 to function as an endotracheal tube, guiding gas through the bladder 320.

Referring in particular to FIG. 3B, the bladder 320 of the EETT 300 is in an expanded state, having been inflated with a gas or filled with another material. In the expanded state, the bladder orifice 335 has been expanded along the length of the bladder 320, here to a consistent a cross-sectional diameter along the entire length of the bladder 320, with the orifice 335 being sufficiently large to provide for acceptable flow of gas to achieve effective ventilation of a subject. The EETT 300 further includes a valve 355 configured to selectively open and close the inlet 345 to allow gas to be provided to the bladder 320 and to help keep gas within the bladder 320 to help ensure sufficient structural stability to have the bladder 320 serve as an endotracheal tube. The valve 355 may be within, or attached to, the air inlet 345 as shown, or alternatively may be part of, or attached to, the bladder 320. One or more eyelets (i.e., one or more Murphy eyes) may be provided at the distal end 325 of the bladder 320.

FIGS. 3C-3D show the cross-sectional area of the bladder 320 in the collapsed, unexpanded state and the expanded state, respectively. As shown in FIG. 3C, a cross-sectional area of the orifice 335 with the bladder 320 in the collapsed state is approximately equal to a cross-sectional area of the body 305, here with an inner diameter 360 of the orifice 335 (i.e., an inner diameter of the bladder 320) being approximately equal to a diameter of the body 305. In this state, an inner surface 370 of the bladder 320 approximates an outer surface of the body 305 and the bladder 320 has an outer diameter 365. As shown in FIG. 3D, a cross-sectional area of the orifice 335 with the bladder 320 in the expanded state is much larger than the cross-sectional area of the body 305, here with an inner diameter 380 of the orifice 335 (i.e., an inner diameter of the bladder 320) being much larger than the diameter of the body 305. In the expanded state, the body 305 may still be attached to the bladder 320. In the expanded state, the inner diameter 380 of the orifice 335, i.e., the inner diameter of the bladder 320, may be larger than the outer diameter 365 of the bladder 320 in the collapsed state to facilitate insertion of the EETT 300 into an ETT already used in a subject (e.g., used to ventilate a patient), e.g., with a diameter of an orifice of the already-used ETT being approximately equal to the inner diameter 380.

Referring to FIGS. 4A-4B, another example EETT 400 includes hinged panels 415, 420, 425. The EETT 400 is configured to change a cross-sectional area, here a cross-sectional diameter 407, by pivoting the panels 415, 420, 425 about respective hinges 410 (with corresponding pivot points). While the EETT 400 is in a collapsed state, each of the hinged panels 425 provides a discrete portion of an outer perimeter of the EETT 400 and the panels 415, 420 are inner panels extending inwardly from the outer perimeter, i.e., from a respective one of the panels 425. The panels 415, 420, 425 can be pivoted with respect to each other about respective ones of the hinges 410 to move the EETT 400 from the collapsed state shown in FIG. 4A to the expanded state shown in FIG. 4B (with a diameter 409 of the EETT 400 being larger in the expanded state than in the collapsed state even though FIGS. 4A-4B are not drawn to scale). By pivoting the panels 415, 420, 425 such that they are staggered in the collapsed state, alternatively spanning a portion of a perimeter (here, essentially a circumference) and extending inwardly (e.g., approximately radially inward) from the perimeter, and (at least some of the panels 415, 420, 425) not staggered in the expanded state, the outer perimeter of the EETT 400 in the compressed state will be less than in an expanded state, with all of the panels 415, 420, 425 forming respective portions of the outer perimeter. For example, if each of the panels 415, 420, 425 provides a similar-length portion of the outer perimeter, and all of the panels 415, 420, 425 form a portion of the outer perimeter in the expanded state, then the outer perimeter in the expanded state may be almost three times longer than the outer perimeter in the collapsed (contracted) state. The panels 425 may be arced to help provide a substantially circular outer perimeter. The panels 415, 420 may be straight, e.g., such that the panels 415, 420 can be disposed in close proximity while in the collapsed state. The panels 415, 420 may, however, be arced to help provide a circular or substantially circular outer perimeter in the expanded state. As shown in FIG. 4B, all of the panels 415, 420, 425 form respective portions of the outer perimeter of the EETT 400 in the fully-expanded state. Expanded states less than the fully-expanded state shown in FIG. 4B are possible, e.g., with fewer than all pairs of the panels 415, 420 pivoted to form parts of the outer perimeter, and/or with one or more pairs of the panels 415, 420 less than fully pivoted such that the hinge 410 between a pair of the panels 415, 420 is disposed inwardly relative to the fully-expanded outer perimeter.

Expansion of the EETT 400 from the compressed state to an expanded state (e.g., from a smaller cross-sectional diameter to a larger cross-sectional diameter) can be facilitated by any number of suitable expansion mechanisms. For example, a physical expander may be disposed in a lumen 440 provided by the EETT 400. Other examples of expansion mechanisms that may be used include shape memory alloys (such as superelastic nitinol), electrical actuator(s), spring bias of the panels 415, 420, and (possibly) 425 toward the fully-expanded state, UV actuated materials (that change shape, e.g., expand, in response to ultraviolet light), a compression sleeve that retains the EETT 400 in the collapsed state while the EETT 400 is biased toward the fully expanded state, means that restrain expansion of the EETT 400 from the inside of the EETT 400, a mechanical expander that can be inserted into the lumen 440 (e.g., a tapered member that pushed the panels 415, 420 toward the fully-expanded state as the tapered member is inserted into the lumen 440), etc.

The hinges 410 may be configured to lock the panels 415, 420, 425 into an expanded orientation, e.g., the fully-expanded state, such that the EETT 400 is inhibited (or even prevented) from returning to the collapsed state. A number of different mechanisms can be used to lock the hinged EETT 400 into an expanded state. For example, referring to FIGS. 4C-4D, an example of the hinge 410 pivotally connects a panel 455 to a panel 460. In FIG. 4C, the panels 455, 460 are in the collapsed state, or an expanded state less than the fully-expanded state, while in FIG. 4D, the panels 455, 460 are in the fully-expanded state and are locked to inhibit further pivoting (e.g., further expansion, or return to a less-expanded state, including the collapsed (fully un-expanded) state). Locking is facilitated by the hinge end of panel 455 sliding into the slot 470 such that further actuation of the hinge is inhibited by the confines of the slot 470. In this manner, once a hinged EETT is expanded, it cannot be contracted unless the hinge end of the inserted panel is removed from the slot in which it is located.

Referring to FIGS. 5A-5B, another example EETT 500 includes an expandable coil 520. In a compressed state, as shown in FIG. 5A, the coil 520 has a cross-sectional diameter 505 of a size that enables insertion of the EETT 500 into a patient's tracheal tube. In this example, the EETT 500 includes a lock 510 that is configured to retain the EETT 500 in the compressed state by inhibiting the coil 520 from expanding until the lock 510 is released, e.g., by a user. The lock 510 can be engaged to retain the coil 520 in an expanded state, as shown in FIG. 5B, inhibiting the coil 520 from collapsing into the compressed state. Alternatively, separate locks may be provided, e.g., one for retaining the coil 520 in the compressed state and one for retaining the coil 520 in an expanded state, e.g., the fully-expanded state.

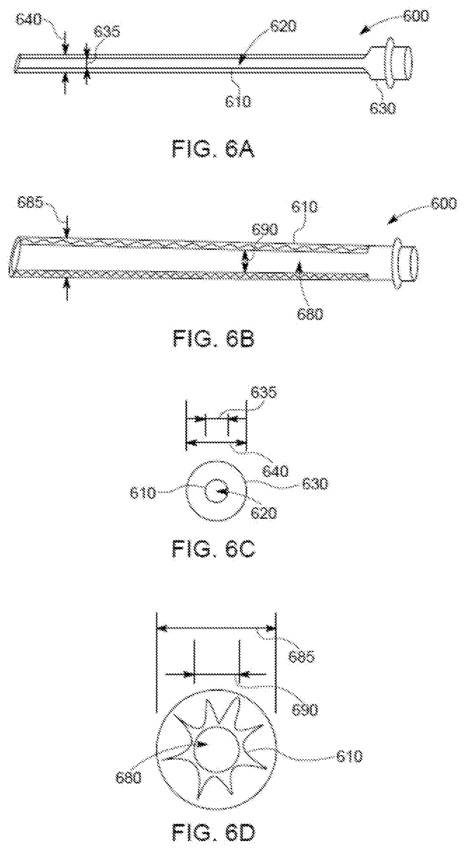

Referring to FIGS. 6A-6D, another example EETT 600 includes an expandable stent 610. In a compressed state, as shown in FIGS. 6A, 6C, the stent 610 is a tubular structure that provides an interior lumen 620 and has an inner diameter 635 (i.e., the diameter of the lumen 620) and an outer diameter 640. The outer diameter 640 may be smaller than a diameter of a lumen of an existing ETT such that the EETT 600 may be inserted inside the lumen of the existing ETT. In an expanded state, as shown in FIGS. 6B, 6D, the stent 610 provides an interior lumen 680 and has an outer diameter 685 and an inner diameter 690 (i.e., the diameter of the lumen 680). The outer diameter 685 of the EETT 600 in the expanded state is such that the EETT 600 will seal a trachea of a subject. The inner diameter 690 of the EETT 600 in the expanded state is such that the EETT 600 will facilitate delivery of oxygen, air, gas or gaseous medicines to the subject and insertion of a replacement EETT as desired.

The EETT 600 further includes a ventilator attachment 630 attached to a distal end of the stent 610. The ventilator attachment 630 may be permanently attached to the stent 610 and of a size allowing removal of another ETT over the stent 610 and the ventilator attachment 630, or may be detachably attached to (i.e., able to be attached to and detached from) the stent 610, e.g., by a user. For example, a detachably attached ventilator attachment 630 may be used if the EETT 600 may be replaced by inserting a replacement ETT into the EETT 600, i.e., into the lumen 620. A detachably attached ventilator attachment (for the EETT 600 or another ETT) may facilitate both swapping an existing ETT for a replacement EETT and connection to a ventilation system.

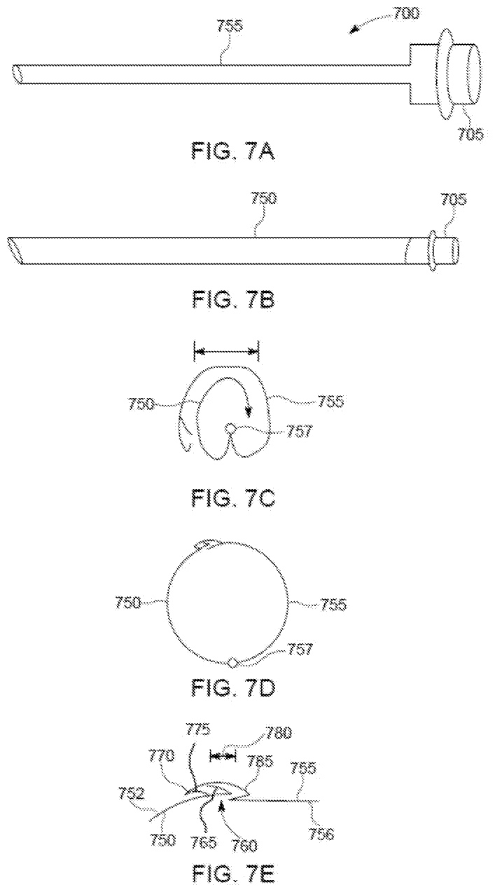

Referring to FIGS. 7A-7D, another example EETT 700 includes an inner tubular section 750 and an outer tubular section 755 pivotally connected to each other by a hinge 757. In a compressed state, as shown in FIGS. 7A, 7C, the tubular sections 750, 755 are nested such that the inner tubular section 750 is disposed inside of the outer tubular section 755. The tubular section 750 may be configured to be flexible (e.g., made of a flexible material and/or of a thickness such that the tubular section 750 can be flexed, e.g., to change a cross-sectional curve of the tubular section from a curve similar to that of the tubular section 755 to a tighter curve, e.g., with a smaller radius) to facilitate nesting with the tubular section 755. The sections 750, 755 can be pivoted with respect to one another to move from the compressed state shown in FIGS. 7A, 7C to an expanded state such as a fully-expanded state shown in FIGS. 7B, 7D. In the expanded states, a cross-sectional area of the EETT 700 is larger than a cross-sectional area of the EETT 700 in the compressed state. The EETT 700 may be configured such that the cross-sectional area of the EETT 700 in the compressed state allows the EETT 700 to be inserted into an ETT that is to be replaced, e.g., that is presently in a subject. The EETT 700 may be configured such that the cross-sectional area of the EETT 700 in an expanded state, such as the fully-expanded state, facilitates the EETT 700 sealing a trachea to facilitate ventilation gas to be provided through the EETT 700 to a subject's lungs. The EETT 700 may include a detachable ventilator connector 705 such as those discussed above.

The EETT 700 may be configured such that, in nested states with the section 750 nested at least partially in the section 755, the section 750 is biased outwardly against the section 755, the section 755 is biased inwardly against the section 750, or the section 750 is biased outwardly and the section 755 is biased inwardly. Pressure applied by one or both of the sections 750, 755 helps a smaller cross-sectional area to be achieved in the compressed state and helps effective locking of the sections 750, 755 in the expanded state.

The EETT 700 may include various locking means to lock the tubular section 750 and the tubular section 755 together in the fully-expanded state (or other expanded state). For example, as shown in FIG. 7E, a mechanical lock 710 includes a hook 760 at an end of the tubular section 750 and a hook 770 at an end of the tubular section 755. The hook 760 includes a barb 765 that is angled outwardly and back toward a main portion 752 of the tubular section 750. The hook 770 includes a barb 775 that is angled inwardly and back toward a main portion 756 of the tubular section 755. The hooks 760, 770 are configured such that the barbs 765, 775 will overlap due to bias of the sections 750, 755 away from each other such that the hooks 760, 770 will nest and interfere with each other, inhibiting further movement of the tubular sections 750, 755 away from each other, thus locking the EETT 700 in the fully-expanded state. The outer tubular section 755 provides an opening 780 of a size which allows the barb 765 to enter the opening 780 thereby enabling meshing of the barb 765 with the barb 775. The design of the hooks 760, 770 capable of meshing with one another in conjunction with the pressure between the tubular sections 750, 755 facilitates a biased system to expand and lock at a predefined cross-sectional diameter. Expansion can be facilitated by a number of different methodologies, including but not limited to, insertion of a physical expander inside the nested sections 750, 755, use of a key which can be turned within the sections 750, 755 thereby rotating the sections 750, 755 relative to each other, use of shape memory alloys (such as superelastic nitinol), electrical actuation, spring bias of the sections 750, 755 with a restriction mechanism which upon removal allows the sections 750, 755 to expand (where the restriction mechanisms can be internal or external to an EETT), magnetic and/or electromagnetic actuated expanders. The second curved section 755 has a third hook 785 positioned such that upon compression of the two curved panels, the hook 785 will accept and secure the hook 760 such that compression is limited to a length predefined by a user. That is, by positioning the third hook 785 at a predefined location, compression and hence reduction of the cross-sectional diameter of the once expanded two piece nested EETT is restricted by the nesting of the hook 760 being nested within the third hook 785.

One or more of the above-described EETTs may include a balloon cuff as discussed with respect to FIG. 1. The balloon cuff may be integral to the EETT or added on as a separate element. The balloon cuff may, however, be omitted as expansion of the various EETTs discussed may make the balloon cuff superfluous.

Still further examples of EETTs may be used. For example, EETTs based on overlapping extended segments or dovetail-like joints capable of a sliding action can be used to achieve desired cross-sectional area expansion. Such systems can be expanded by a force, such as a force produced by a piston drive mechanism, by air under pressure, by mechanical actuation, or by spring action. In yet other examples, suitable EETTs can be based on spaced segments with actuating members filling the spaces between adjacent segments where the segments have curved edges that are actuated by a force from, e.g., a syringe type mechanism, air pressure, spring pressure, etc., such that sliding movement of the actuating members will cause the circumference of the ETT to expand or contract. In yet another embodiment, an EETT based upon a coiled spring has one end secured to a rod whose distal end is inserted to a predefined insertion depth and whose proximal end is secured such that the rod does not move relative to the predefined ETT insertion depth. Upon un-coiling of the spring, a cross-sectional area of the ETT expands to a tracheal lumen area while the distal end of the rod does not pass a user defined insertion depth.

Another example of an EETT includes a stent-like infrastructure that runs substantially longitudinally along a length of a tubular member. The stent-like infrastructure is configured to variably expand a cross-sectional area bounded by the inner surface of the tubular member. Furthermore, the stent-like infrastructure is arranged to maintain the variable expansion of the cross-section to substantially prevent stenosis of the body lumen, while the body is ventilated via the EETT. Such stent-like structures can be actuated by a number of means as is known in the art associated with stents.

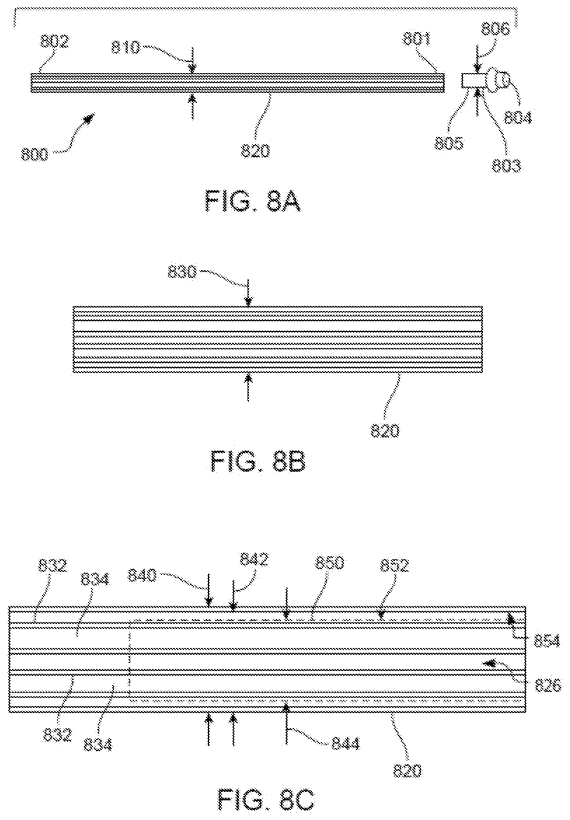

Referring to FIGS. 8A-8C, an example of an EIC 800 includes a body 820 having a proximal end 801 and a distal end 802. The EIC 800 is configured to be expandable/contractable such that the EIC 800 may change between an expanded (larger) state and a contracted (smaller state). In at least the expanded state (and perhaps one or more intermediate states between the smaller state and the expanded state), the EIC 800 may receive an ETT through the EIC 800 to facilitate insertion of the ETT into a patient.

For example, the EIC 800 is capable of having a cross-sectional area expanded from a smaller cross-sectional area to a larger cross-sectional area, e.g., with an outer diameter changing from a contracted diameter 810 to an intermediate diameter 830 to a fully-expanded diameter 840 with corresponding inner diameters. The EIC 800 is configured to change between a smaller-size state (e.g., an insertion state that may facilitate insertion) as shown in FIG. 8A and a larger-size state (e.g., an expanded state that may facilitate intubation) as shown in FIG. 8C with intermediate states, one of which is shown in FIG. 8B, in between. For example, the advancement (e.g., insertion) of an ETT 850 in an interior of the body 820 of the EIC 800 may cause an expansion of the body 820 of the EIC 800 until the body 820 is restricted by an inner diameter of a tracheal mucosa of a patient. The EIC 800 in this example includes stiffening rods 832 connected by membranes 834 that will inhibit expansion of the EIC 800, but in this example at a cross-sectional area larger than the tracheal mucosa of a patient. An outer surface 852 of the ETT 850 may push against an inner surface 854 of the body 820 to cause an inner diameter 842 of the body 820 to expand to at least an outer diameter 844 of the ETT 850.

The smaller cross-sectional area may be of a size and shape to facilitate the EIC 800 fitting within an ETT already located in the tracheal lumen of a patient and used to ventilate the patient. For example, the cross-sectional area in the smaller state may be less than 60 mm², e.g., about 50 mm² or less for use with an adult male, about 40 mm² or less for use with an adult female, about 5-7 mm² or less for use with an infant, or about 3-5 mm² or less for use with a premature baby. For a circular cross section, the contracted diameter 810 may be smaller than an inner diameter of a standard ETT (e.g., about 8 mm for an ETT for an adult male, about 7 mm for an ETT for an adult female, about 2.5-3 mm for an infant, about 2-2.5 mm for a premature baby, although these are merely examples and the size used depends on each individual). Once the EIC 800 is introduced in the ETT, the ETT may be removed, leaving the EIC 800 in the tracheal lumen. Following removal of the ETT, the EIC 800 may be expanded to a larger cross-sectional area, e.g., having the expanded diameter 840, such that the EIC 800 may provide a seal with tracheal walls of the patient and may provide a lumen 826, shown in FIG. 8C with the EIC 800 in the expanded state such that the lumen 826 is amenable to ventilation of the patient. With the EIC 800 in the expanded state, the inner diameter 842 of the EIC 800 is at least as large as the external diameter 844 of the ETT 850 received by the body 820 of the EIC 800.

With particular reference to FIG. 8C, the EIC 800, an in particular the body 820, is in a fully-expanded state, expanded by the forward movement of the ETT 850 within the body 820. The body 820 has been fully expanded to have an expanded outer diameter 840.

Referring in particular to FIG. 8A, the proximal end 101 of the body 820 is configured (e.g., sized, shaped, etc.) to detachably receive a ventilator connector 803 that is configured to be attachable/detachable to/from the proximal end 101. The ventilator connector 803 has a proximal end 804 and a distal end 805. The distal end 805 has an outer diameter 806 (and corresponding inner diameter) sized to receive the proximal end 101 of the body 820. The ventilator connector 803 may be detachably attached to (i.e., able to be attached to and detached from) the EIC 800, e.g., by a user. For example, the ventilator attachment 803 may be used if the EIC 800 may be replaced by inserting a replacement ETT into the EIC 800, e.g., into the lumen 826, shown in FIG. 8C with the EIC in the contracted state. The ventilator connector 803 being configured for detachable attachment may facilitate both swapping an existing ETT for a replacement ETT and connection to a ventilation system. Alternatively, the ventilator connector 803 may be permanently attached to the EIC 800 and of a size allowing for removal of an ETT.

The EIC 800 may include a balloon cuff, e.g., similar to the cuff 140 shown in, and discussed with respect to, FIG. 1. The balloon cuff may be integral to the EIC 800 or added on as a separate element. The balloon cuff may, however, be omitted as expansion of the EIC 800 may make the balloon cuff superfluous.

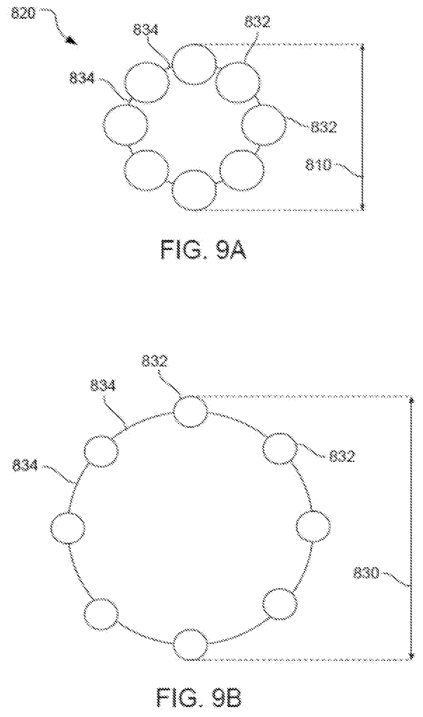

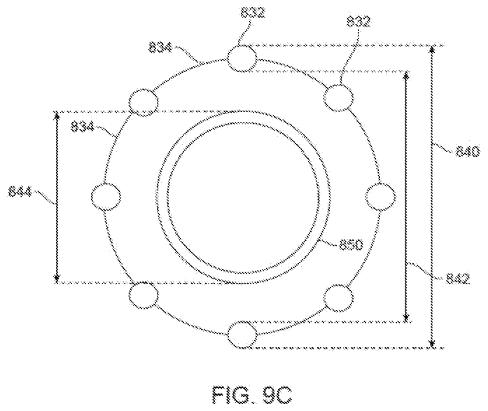

Referring to FIGS. 9A-9C, shown are cross-sectionals view of the EIC 800 having the external diameter 810 in the contracted (or compressed) state, the outer diameter 830 in an intermediate state, and the outer diameter 840 in the expanded state. The cross-sectional views show that the EIC 800 includes the stiffening rods 832 with flexible membranes 834 between and connected each pair of adjacent stiffening rods 832. The membranes 834 may be connected to the stiffening rods 832 in a variety of ways, e.g., connected to outsides of the rods 832 and/or connected to insides of the rods 832, bonded to the rods 832, disposed between layers forming the membranes 834, etc.

Referring in particular to FIG. 9B, the EIC 800 is in a partially-expanded state with the outer diameter 830 being larger than the outer diameter 810 of the EIC in the contracted state. The flexible membranes 834 stretch between the stiffening rods 832 to allow expansion of the cross-sectional area of the EIC 800, here allowing enlargement of the inner and outer diameters of the EIC 800.

Referring in particular to FIG. 9C, the EIC 800 is in the fully-expanded state with the outer diameter 840 being larger than the diameter 830, here at a maximum diameter for the EIC 800. The flexible membranes 834 between the stiffening rods 832 are fully stretched. The fully-expanded diameter 840 allows introduction of the ETT 850 into the lumen 826 of the EIC 800 with the external diameter 844 of the ETT 850 being smaller than the internal diameter 842 of the EIC 800.

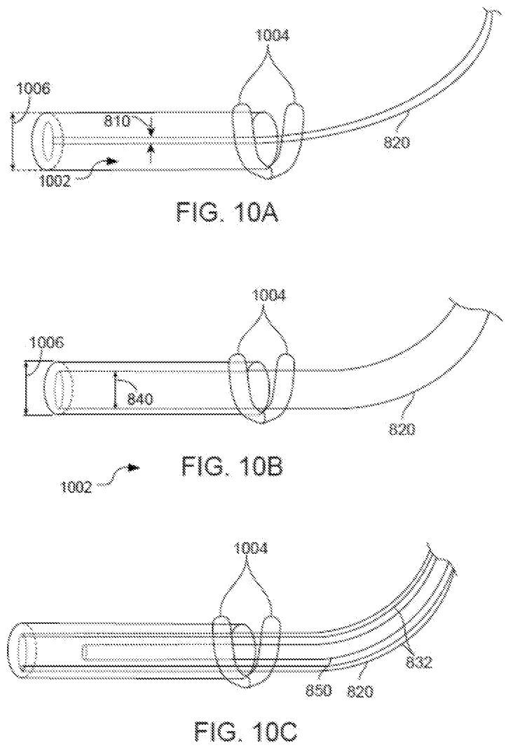

Referring to FIGS. 10A-10C, illustrated here is the the EIC 800 within a tracheal lumen 1002 of a patient as the EIC 800 is passed through vocal cords 1004 of the patient. Referring in particular to FIG. 10A, the EIC 800 is in the contracted state, with the body 820 having the outer diameter 810. Referring in particular to FIG. 10B, the EIC 800 is in the fully-expanded state with the outer diameter 840 being smaller than an inner diameter 1006 of the trachea of the patient. Referring in particular to FIG. 10C, the ETT 850 has been introduced into, received by, and is disposed in, the lumen 826 of the fully-expanded EIC 800. The lumen 826 of the EIC 800 expands from the proximal end 101 of the body 820 to the distal end 102 of the body 820 as the ETT 850 is advanced through the body 820, through the vocal cords 1004 into the tracheal lumen 1002.

The ETT 850 may be inserted into the lumen 826 of the EIC 800 to cause the EIC 800 to expand across the section of the trachea filling out the tracheal lumen 1006. The stiffening rods 832 of the EIC 800 are configured to guide the ETT 850 as the ETT 850 is inserted into the EIC 800 and received by the lumen 826. The stiffening rods 832 may be configured to help prevent a lateral deflection of the ETT 850 as the ETT comes in contact with a more narrow section of the vocal cords 1004. This may prevent the ETT 850 from getting caught or hung up at the level of the vocal cords 1004.

Expansion of the EIC 800 from the compressed state to the expanded state (e.g., from a smaller cross-sectional diameter to a larger cross-sectional diameter) may be facilitated by one or more suitable expansion mechanisms. For example, a physical expander may be disposed in the lumen 826 of the EIC 800. In most cases, this physical expander is the ETT 850 but other expanders may be used. Other examples of expansion mechanisms that may be used include shape memory alloys (such as super-elastic nitinol), electrical actuator(s), spring bias of the membranes 834 toward the fully-expanded state, UV-actuated materials (that change shape, e.g., expand, in response to ultraviolet light (UV)).

Other Considerations

Other examples and implementations are within the scope and spirit of the disclosure and appended claims. Substantial variations may be made in accordance with specific requirements.

As used herein, “or” as used in a list of items prefaced by “at least one of” or prefaced by “one or more of” indicates a disjunctive list such that, for example, a list of “at least one of A, B, or C,” or a list of “one or more of A, B, or C,” or “A, B, or C, or a combination thereof” means A or B or C or AB or AC or BC or ABC (i.e., A and B and C), or combinations with more than one feature (e.g., AA, AAB, ABBC, etc.).

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of operations may be undertaken before, during, or after the above elements are considered. Accordingly, the above description does not bound the scope of the claims.

What is claimed is:

1. An expandable endotracheal device comprising:
an elongated body configured to change from smaller-size state to a larger-size state;
wherein in the smaller-size state, a perimeter, and thus a cross-sectional area, of the elongated body is restricted to facilitate insertion of the elongated body within a passageway of an in-use endotracheal tube; and
wherein the elongated body is configured to:
have at least a portion of the perimeter of the elongated body expand to seal to a trachea of a subject; or
provide an inner passageway extending through the elongated body along a length of the elongated body and with a boundary defining the inner passageway being configured to slidably receive a ventilation mechanism; or
a combination thereof.

2. The expandable endotracheal device of claim 1, wherein the elongated body provides, in the larger-size state, the inner passageway sufficiently sized for conveying fluid to lungs of the subject.

3. The expandable endotracheal device of claim 2, wherein the elongated body has a cross-sectional area of less than 60 mm² in the smaller-size state.

4. The expandable endotracheal device of claim 1, wherein the elongated body includes: (1) an inflatable bladder, (2) a stent, (3) a coil, (4) a shape-memory alloy, (5) a light-activated shape-changing material, (6) a mechanical expander, (7) an expandable material, or (8) a compression sleeve, or a combination of any of items (1)-(8).

5. The expandable endotracheal device of claim 1, wherein the elongated body is configured to provide different outward pressures at different positions along the length of the elongated body and/or to have different outer perimeter sizes at different positions along the length of the elongated body.

6. The expandable endotracheal device of claim 1, wherein the at least a portion of the perimeter of the elongated body is configured to adapt to a shape of a wall of the trachea of the subject.

7. The expandable endotracheal device of claim 1, wherein a distal end portion of the elongated body is curved along a length of the distal end portion.

8. The expandable endotracheal device of claim 1, wherein the elongated body includes a stent.

9. The expandable endotracheal device of claim 1, further comprising a detachable ventilator connector detachably connected to a proximal end of the elongated body and configured to be connected to a ventilation system.

10. The expandable endotracheal device of claim 1, wherein the elongated body is configured to provide the inner passageway extending through the elongated body along the length of the elongated body and with the boundary defining the inner passageway being configured to slidably receive a ventilation mechanism, and wherein the elongated body comprises a plurality of rods extending lengthwise along the elongated body.

11. The expandable endotracheal device of claim 10, wherein the elongated body comprises a plurality of flexible membranes each connected to a pair of the rods along lengths of the rods in the pair of rods.

12. An expandable endotracheal device comprising:
means for conveying gas from a ventilation system to a lung of a subject; and
means for altering at least one of a size or a shape of at least a portion of an outer perimeter of the means for conveying gas from a smaller-size state, in which the outer perimeter is sized and shaped to slide within a passageway of an in-use endotracheal tube, to a larger-size state in which at least a portion of the outer perimeter will seal a trachea of a subject.

13. The expandable endotracheal device of claim 12, wherein the means for conveying gas are for providing an inner cavity through the means for conveying and sufficiently sized for conveying ventilation gas to lungs of the subject.

14. The expandable endotracheal device of claim 12, wherein the means for altering cause the means for conveying gas to have a cross-sectional area of less than 60 mm² in the smaller-size state.

15. The expandable endotracheal device of claim 12, wherein the means for altering include: (1) an inflatable bladder, (2) a stent, (3) a coil, (4) a shape-memory alloy, (5) a light-activated shape-changing material, (6) a mechanical expander, (7) an expandable material, or (8) a compression sleeve, or a combination of any of items (1)-(8).

16. The expandable endotracheal device of claim 12, wherein the means for altering are for providing different outward pressures at different positions along a length of the means for conveying and/or for providing different outer perimeter sizes at different positions along the length of the means for conveying.

17. The expandable endotracheal device of claim 12, wherein the means for altering are for adapting the at least a portion of the outer perimeter to a shape of a tracheal wall of the subject.

18. The expandable endotracheal device of claim 12, wherein a distal end portion of the means for conveying is curved along a length of the distal end portion.

19. The expandable endotracheal device of claim 12, wherein the means for altering include a stent.

20. The expandable endotracheal device of claim 12, wherein the means for conveying comprise a tube and a connector detachably connected to the tube and configured to be connected to the ventilation system.

21. The expandable endotracheal device of claim 12, wherein the means for conveying gas include a plurality of rods extending along a length of the means for conveying gas and a plurality of flexible membranes each attached to a pair of the plurality of rods.

22. The expandable endotracheal device of claim 12, wherein the means for altering at least one of the size or the shape of at least the portion of the outer perimeter of the means for conveying gas are configured to alter at least one of the size or the shape of at least the portion of the outer perimeter of the means for conveying gas in response to receipt of a member in a cavity defined by the means for conveying gas.
